SEQUENCE LISTING (16 sequences)

SEQ ID NO: 1 - protein, 7 aa, artificial sequence
  The amino acid sequence of the oligopeptide
KHGHHRH

SEQ ID NO: 2 - protein, 9 aa, artificial sequence
  The amino acid sequence of the oligopeptide with cysteines at both ends
CKHGHHRHC

SEQ ID NO: 3 - DNA, 2768 bp, artificial sequence
  optimized DENV-2 NS5 gene
ataattttgtttaactttaagaaggagatatacatatgggtaccggtaatattggcgaaaccctgggcgaaaagtggaaaatccgcctgaacgcactgggcaaaagcgagttccagatctacaagaagagcggtattcaggaagttgatcgtaccctggcgaaagaaggcattaaacgcggcgaaaccgatcatcacgcagttagtcgcggtagcgcaaaactgcgttggtttgtcgagcgcaacatggttaccccggaaggcaaagttgttgatctgggttgcggtcgcggcggttggtcttattattgcggtggcctgaaaaacgttcgcgaagttaaaggtctgaccaaaggcggtccgggtcacgaagaaccgattccgatgagtacctacggttggaatctggttcgtctgcagtctggcgttgacgttttctttaccccgccggaaaaatgcgataccctgctgtgcgatattggcgaaagtagtccgaatccgaccgttgaagcaggtcgtaccctgcgcgttctgaatctggttgaaaactggctgaacaacaacacccagttctgcatcaaggttctgaacccgtatatgccgagcgttatcgagaagatggagaccctgcaacgcaaatacggtggtgcactggttggtaatccgctgagtcgtaactccacccacgaaatgtactgggttagcaacgcgagcggcaatattgtttcctccgtcaacatgatctcccgcatgctgatcaaccgctttaccatgcgccataagaaagcgacctacgaaccggacgttgatctgggttctggtacccgtaacattggcatcgaaagcgaaatcccgaatctggatatcatcggcaaacgcatcgagaagatcaagcaggagcacgaaaccagttggcattacgatcaggaccatccgtacaaaacctgggcatatcacggcagctacgaaaccaaacagaccggttctgcaagcagtatggttaacggcgttgttcgtctgctgaccaaaccgtgggacgttgttccgatggttacccaaatggcaatgaccgataccaccccgtttggtcagcagcgcgttttcaaagagaaggtcgatacccgtacccaagaaccgaaagaaggcaccaagaagctgatgaagatcaccgctgagtggctgtggaaagaactgggcaagaagaaaaccccgcgtatgtgtacccgcgaagaattcacccgtaaagttcgtagtaacgctgcactgggtgcgattttcaccgacgaaaacaagtggaagtctgcacgcgaagcagttgaagatagtcgtttctgggagctggtcgacaaagaacgtaacctgcatctggaaggtaagtgcgaaacctgcgtctacaacatgatgggcaaacgcgagaagaaactgggcgaatttggcaaagcgaaaggcagtcgcgctatttggtatatgtggctgggcgcacgttttctggaatttgaagcactgggcttcctgaacgaagatcactggtttagccgcgaaaacagtctgtctggcgttgaaggcgaaggtctgtataaactgggctatatcctgcgcgatgtcagcaaaaaagaaggcggcgcaatgtatgcagacgataccgcaggttgggatacccgtattaccctggaagacctgaagaacgaagaaatggtcaccaaccacatggaaggcgaacacaagaaactggcggaagcgatcttcaagctgacctaccagaacaaagtcgttcgcgttcaacgtccgaccccgcgcggtaccgttatggatattattagccgtcgcgatcaacgcggttctggtcaagttggtacctacggtctgaacaccttcaccaacatggaagcgcagctgattcgtcagatggaaggcgaaggcgtattcaaaagcatccagcatctgaccgttaccgaagaaattgcggttcaaaattggctggcacgcgttcgtcgcgaacgtctgtctcgtatggcaatttctggcgacgattgcgtagttaaaccgctggatgatcgttttgcatctgcactgaccgctctgaacgatatgggcaaagtccgcaaagacattcaacagtgggaaccgagtcgcggttggaacgattggacccaagttccgttttgcagccatcacttccacgagctgatcatgaaagacggtcgcgttctggtagttccgtgtcgtaatcaagacgaactgattggtcgcgcacgtatttctcaaggcgcaggttggtcactgcgcgaaaccgcttgtctgggtaaatcttacgcacagatgtggagcctgatgtactttcatcgtcgcgatctgcgtctggcagcaaacgcgatttgttctgcagttccgagtcattgggttccgaccagtcgtaccacctggagtattcacgccaaacacgagtggatgaccaccgaagatatgctgaccgtatggaaccgcgtttggatccaagaaaacccgtggatggaagacaaaaccccggttgaaagctgggaagaaatcccgtatctgggtaaacgcgaagatcagtggtgcggtagtctgattggtctgacctctcgcgcaacctgggcaaaaaacatccagaccgcgatcaaccaggtccgtagcctgattggcaacgaagagtataccgactacatgccgagcatgaaacgctttcgtcgcgaagaagaagaagctggcgtactgtggcatcatcatcatcatcactaatgaaagctt

SEQ ID NO: 4 - protein, 907 aa, artificial sequence
  the amino acid sequence encoding the optimized DENV-2 NS5 gene
MGTGNIGETLGEKWKIRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFTPPEKCDTLLCDIGESSPNPTVEAGRTLRVLNLVENWLNNNTQFCIKVLNPYMPSVIEKMETLQRKYGGALVGNPLSRNSTHEMYWVSNASGNIVSSVNMISRMLINRFTMRHKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLYKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTVTEEIAVQNWLARVRRERLSRMAISGDDCVVKPLDDRFASALTALNDMGKVRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQTAINQVRSLIGNEEYTDYMPSMKRFRREEEEAGVLWHHHHHH

SEQ ID NO: 5 - protein, 4 aa, artificial sequence
  tetrapeptide
KHGH

SEQ ID NO: 6 - protein, 4 aa, artificial sequence
  tetrapeptide
HGHH

SEQ ID NO: 7 - protein, 4 aa, artificial sequence
  tetrapeptide
GHHR

SEQ ID NO: 8 - protein, 4 aa, artificial sequence
  tetrapeptide
HHRH

SEQ ID NO: 9 - protein, 5 aa, artificial sequence
  pentapeptide
KHGHH

SEQ ID NO: 10 - protein, 5 aa, artificial sequence
  pentapeptide
HGHHR

SEQ ID NO: 11 - protein, 5 aa, artificial sequence
  pentapeptide
GHHRH

SEQ ID NO: 12 - protein, 6 aa, artificial sequence
  hexapeptide
KHGHHR

SEQ ID NO: 13 - protein, 6 aa, artificial sequence
  hexapeptide
HGHHRH

SEQ ID NO: 14 - DNA, 17 bp, artificial sequence
  96gIII sequencing primer
ccctcatagtcgtaacg

SEQ ID NO: 15 - DNA, 21 bp, artificial sequence
  codon sequence
aagaatactcttcatacgttt

SEQ ID NO: 16 - DNA, 21 bp, artificial sequence
  codon sequence
aagcatggtcatcatcgtcat

(19) United States
(12) Patent Application Publication
(10) Pub. No.: US 2021/0040153 A1
(43) Pub. Date: Feb. 11, 2021

(54) OLIGOPEPTIDE HAVING DENGUE VIRUS REPLICATION INHIBITION FUNCTION AND APPLICATION THEREOF

(71) Applicant: Beijing University of Technology, Beijing (CN)

(72) Inventors: Minglian Wang, Beijing (CN); Jinsong Li, Beijing (CN); Qun Wang, Beijing (CN); Yishu Yang, Beijing (CN); Jintao Li, Beijing (CN); Wei Liu, Beijing (CN); Xiangqian Xiao, Beijing (CN); Xiaojin Su, Beijing (CN)

(21) Appl. No.: 16/959,669

(22) PCT Filed: Dec. 28, 2018
(86) PCT No.: PCT/CN2018/124985

§ 371 Date: Jul. 1, 2020

(30) Foreign Application Priority Data
Jan. 10, 2018 (CN) 201810022857.1

(51) Int. Cl. C07K 7/06; C07K 5/09; C07K 5/11

(52) U.S. Cl. CPC C07K 7/06; A61K 38/00; C07K 5/1019; C07K 5/0815

(57) ABSTRACT

The present invention relates to the field of virology, and specifically discloses a short peptide having a dengue virus replication inhibition function and an application thereof. The amino acid sequence of the short peptide provided in the present invention is KHGHHRH, i.e. Lys-His-Gly-His-His-Arg-His (SEQ ID NO. 1). The short peptide has a high specificity affinity with NS5 and has the function of efficiently inhibiting dengue virus replication, the anti-viral effect thereof not been limited to DENV-2, but also having a significant inhibitory effect on the replication of type 1, type 3, and type 4 dengue virus. One cysteine is added to the two ends of the short peptide sequence, the short peptide being cyclised by means of the cysteines at the two ends to form a cyclic peptide. The obtained cyclic peptide strengthens the dengue virus replication inhibition function, and can be used for specific treatment of dengue virus infection.

### CROSS REFERENCE TO RELATED APPLICATIONS

This application claims priority to Chinese Patent Application No. 201810022857.1 entitled “SHORT PEPTIDE HAVING DENGUE VIRUS REPLICATION INHIBITION FUNCTION AND APPLICATION THEREOF” on Oct. 1, 2018, the entire contents of which are incorporated herein by reference.

### TECHNICAL FIELD

The invention relates to the field of virology, in particular to a short peptide having the effect of inhibiting dengue virus replication obtained by using genetic engineering and phage display peptide library technology.

### BACKGROUND

Demme virus (DENV) can cause dengue fever, dengue hemorrhagic fever, and dengue shock syndrome. It is widely prevalent in tropical and subtropical regions. It is the most widely distributed, most frequently occurring, and most harmful infectious disease. Each year, about 390 million people are infected worldwide, and nearly 100 million people show symptoms of infection, most of which are dengue fever cases, and there are more than 500,000 cases of dengue hemorrhagic fever and dengue shock syndrome. The average annual death rate due to dengue virus infection is more than 22,000 cases, mostly children (World Health Organization, 2009; Bhatt et al.., 2013; Guzman and Harris, 2015). Infectious diseases caused by dengue virus infection have caused serious harm in many countries in Asia, the Pacific Islands, and Central and South America. In China, it has also changed from imported and sporadic diseases to perennial diseases. In southern areas such as Taiwan, Hong Kong and Guangdong, it is perennially epidemic. For example, in 2014, there were 40,000 cases in one epidemic in Guangzhou alone.

DENV belongs to the family Flaviviridae, genus Flavivirus. It is divided into four stereotypes, DENV 1 to 4, which can cause pathogenicity to humans. Among these, DENV 2 is the most widely transmitted stereotype, and the severe rate and death after infection are also higher than other types. After a dengue fever outbreak in Malaysia in recent years, virologist Nikos et al, at the University of Texas, Medical Center, in the United States isolated a virus strain and sequenced the whole genome to find out that it is a new type of dengue virus. Whether this Malay dengue virus will continually spread and become epidemic among people is unknown (Nonnile D. 2013.Science.342:415), and whether to define it as DENV5 remains to be explored.

DENV is transmitted by female mosquitoes, mainly Aedes aegypti and Aedes athopictus.After a female mosquito bites a DENV-infected person, the virus proliferates in the mosquito and causes the spread of the virus and the infection of those Who are bitten by the mosquito. The population is more sensitive to the primary infection. of any type of DENV. After infection, they acquire immunity to homovirus for 1 to 4 years, but immunity to heterotypic viruses is very short, which only lasts 2 to 12 months. Therefore, a secondary or continuous infection may occur after infection with one type of DENV, and the incidence and mortality of dengue hemorrhagic fever and dengue shock syndrome caused by secondary heterotypic infections are higher. This is because pre-existing cross-antibodies can bind to the virus in secondary heterotypic infections, and promote the infectivity of target cells including monocytes, macrophages and mature dendritic cells through the interaction of the antibodies with Fe receptors on the surface of the target cells, thereby causing clinical symptoms such as blood concentration, bleeding or hemafecia, or even shock. Patients with dengue hemorrhagic fever are similar to patients with dengue fever in the fever stage, but the physical signs of the patients rapidly worsen after the fever. Bleeding symptoms appear, and even hypovolemic shock occurs. The course of disease is shorter, but the disease is more fatal. This antibody-dependent enhancement (ADE) after secondary infection is a characteristic of the pathogenicity of DENV infection, and it is also the main obstacle for the development of viral vaccines. That is because if the vaccine does not produce sufficient protective antibodies against all types of viruses, it will aggravate the infection of the heterotypic virus. Moreover, vaccines may be discarded as new types of viruses emerge. Therefore, shortly after the French pharmaceutical company Sanofi Pasteur launched the world's first chimeric dengue fever quadrivalent vaccine in Mexico and the Philippines in January 2016 after decades of research and development, the safety and protection have been jointly warned by the World Health Organization and several countries where the vaccine had been already used. Especially among children, those who have been vaccinated with the vaccine are more likely to develop severe dengue fever than children who have never been vaccinated (WHO, 2017; Aguiar et al., 2016; Flasche et al., 2016; Halstead, 2016; Halstead and Russell, 2016; Wilder-Smith et al., 2016), the dawn that people just saw immediately dims.

At present, DENV infection is limited to symptomatic treatment, and there are no specific and effective antiviral drugs. It is expected that effective antiviral targets can be selected according to the structure of the viral genome and th.e function of the encoded protein. DENV is a coated single positive-stranded RNA virus with an icosahedral structure. The diameter of the virion is 45-55 nm and the genome size is 10.7 kb. The viral genome is infectious and can be used directly as mRNA to initiate translation of viral proteins. Its genome encodes about 3300 amino acids, forming a polyprotein precursor molecule, which is cleaved into 3 structural proteins and 7 non-structural proteins by the combined action of virus and host protease. The ¼ sequence at the 5′ end of the genome thereof encodes the structural proteins of the virus, and participates in the process of virus life cycle, such as virus and cell adsorption, virus entry into cells, cell membrane fusion, virus assembly and the like. It can stimulate the body to produce protective antibodies, but it is also the main cause that leads to the ADE effect. The ¾ sequence at the 3′ end encodes the non-structural proteins (NS), which performs functions such as viral genome replication, post-translational processing of viral proteins, intracellular signal transduction and the like. Among these, NS5 is the largest protein (104kD) encoded by the DENV genome, and it is also the most conservative. Its main function is the function of RNA-dependent RNA polymerase (RdRp), which is responsible for the RNA replication of the viral genome. There is no homologue of RdRp in normal host cells, so it can be used to screen DENV inhibitors in vitro; and since there is no similar structure protein in host cells, this protein inhibitor will have better virus specificity. Therefore, NS5 protein has become the main target of antiviral drug research in recent years.

However, due to the large size of the protein, the full-length expression of the protein is difficult. After the functional region thereof is expressed, it is difficult to form the characteristic conformation thereof and the protein loses its function. Therefore, it is urgent to develop a method which can express the full-length NS5 protein and form the characteristic conformation, and on this basis, further develop effective antiviral drugs.

### SUMMARY

In order to solve the problems existing in the prior art, an object of the disclosure is to provide an oligopeptide having an inhibitory effect on dengue virus replication.

In order to achieve the object of the disclosure, the technical solution of the disclosure is as follows:

DENV 2 is the most widely transmitted stereotype, and the severity and mortality after infection are also higher than other types. In this disclosure, the DENV-2 NS5 gene is codon-optimized, and then a full-length DENV NS5 expression system is constructed. By optimizing the induction conditions, a full-length DENV NS5 recombinant protein is obtained. After purification of the recombinant protein, it is coated as a target molecule and screened in accordance with the conformational peptide library displayed by phage to obtain several short peptides with high affinity to NSS, and they are sequenced. It has been found in cell poisoning experiments that one conformational short peptide of the several short peptides has a significant inhibitory effect on the replication of DENV 2 virus, showing a highly effective antiviral effect. After using it in experiments on other serotypes of dengue virus, it has been found that the antiviral effect of this oligopeptide is not limited to DENV-2, but also has a significant inhibitory effect on the replication of dengue viruses of types 1, 3 and 4.

The disclosure provides an oligopeptide that has the function of inhibiting the dengue virus replication and has an amino acid sequence of KHGHHRH, that is, Lys-His-Gly-His-His-Arg-His.

The oligopeptide has high specific affinity for NS5, can effectively inhibit the replication of dengue virus, and can be used for specific treatment of dengue virus infection.

Further, the disclosure also provides the application of the oligopeptide in the manufacture of a medicament for treating dengue virus infection, and the application of the oligopeptide in the manufacture of a medicament for inhibiting dengue virus replication.

It should be noted that a pharmaceutical composition containing the oligopeptide of the disclosure also belongs to the protection scope of the disclosure.

Alternatively, the oligopeptide may be cyclized to fonn a cyclic peptide. For example, cysteines can be synthesized at the two ends of the oligopeptide to cyclize the oligopeptide, or the oligopeptide can be cyclized by means of forming an amide bond ring by the carboxyl group and the N-terminal amino group of the middle side chain of the heptapeptide sequence, forming an amide ring by the amino group and the C-terminal carboxyl group of the side chain, forming a ring by the head and tail amides of the heptapeptide molecule and the like. The obtained cyclic peptide has the effect of effectively inhibiting dengue virus replication, and will be used for specific treatment of dengue virus infection.

Moreover, it also has been found in this disclosure that the tripeptide, tetrapeptide. pentapeptide or hexapeptide fragment in the oligopeptide, such as KHG, HGH, GHH, HHR, HRH, KHGH, HGHH, GHHR, HHRH, KHGHH, HGHHR, GHHRH KHGHHR and HGHHRH, also have high specific affinity for NS5.

Further, the disclosure also provides an application of the above tripeptide, tetrapeptide, pentapeptide or hexapeptide fragment in the manufacture of a medicament for treating dengue virus infection and a medicament for inhibiting dengue virus replication.

Furthermore, a pharmaceutical composition containing the above tripeptide, tetrapeptide pentapeptide or hexapeptide fragment also belongs to the protection scope of this disclosure.

The oligopeptides of the disclosure are obtained by screening using the following steps:

1. Codon Optimization of DENV NS5 Gene

The DENV NS5 gene sequence is from NCBI GenBank (Accession number: AF038403.1), and the codon is optimized using the MaxCodon™ Optimization Program.

The Nde I restriction site (5′-CATATG-3′) is added at the 5′ end of the optimized sequence, and a coding sequence encoding 6 histidines and Hind 111 restriction site sequence (5′-AAGCTT-3′) are added at the 3′ end (see bold letters). Detai Bio-Tech (Nanjing) Co., Ltd. was commissioned to synthesize the following sequence (the underlined is the restriction site, and the preceding number is the nucleotide number after subsequent insertion into the plasmid vector):

| | |
|---|---|
| 5041 | ATAATTTTGT TTAACTTTAA GAAGGAGATA TACATATGGG TACCGGTAAT ATTGGCGAAA |
| 5101 | CCCTGGGCGA AAAGTGGAAA ATCCGCCTGA ACGCACTGGG CAAAAGCGAG TTCCAGATCT |
| 5161 | ACAAGAAGAG CGGTATTCAG GAAGTTGATC GTACCCTGGC GAAAGAAGGC ATTAAACGCG |
| 5221 | GCGAAACCGA TCATCACGCA GTTAGTCGCG GTAGCGCAAA ACTGCGTTGG TTTGTCGAGC |
| 5281 | GCAACATGGT TACCCCGGAA GGCAAAGTTG TTGATCTGGG TTGCGGTCGC GGCGGTTGGT |
| 5341 | CTTATTATTG CGGTGGCCTG AAAAACGTTC GCGAAGTTAA AGGTCTGACC AAAGGCGGTC |
| 5401 | CGGGTCACGA AGAACCGATT CCGATGAGTA CCTACGGTTG GAATCTGGTT CGTCTGCAGT |
| 5461 | CTGGCGTTGA CGTTTTCTTT ACCCCGCCGG AAAAATGCGA TACCCTGCTG TGCGATATTG |
| 5521 | GCGAAAGTAG TCCGAATCCG ACCGTTGAAG CAGGTCGTAC CCTGCGCGTT CTGAATCTGG |
| 5581 | TTGAAAACTG GCTGAACAAC AACACCCAGT TCTGCATCAA GGTTCTGAAC CCGTATATGC |
| 5641 | CGAGCGTTAT CGAGAAGATG GAGACCCTGC AACGCAAATA CGGTGGTGCA CTGGTTGGTA |
| 5701 | ATCCGCTGAG TCGTAACTCC ACCCACGAAA TGTACTGGGT TAGCAACGCG AGCGGCAATA |
| 5761 | TTGTTTCCTC CGTCAACATG ATCTCCCGCA TGCTGATCAA CCGCTTTACC ATGCGCCATA |
| 5821 | AGAAAGCGAC CTACGAACCG GACGTTGATC TGGGTTCTGG TACCCGTAAC ATTGGCATCG |
| 5881 | AAAGCGAAAT CCCGAATCTG GATATCATCG GCAAACGCAT CGAGAAGATC AAGCAGGAGC |
| 5941 | ACGAAACCAG TTGGCATTAC GATCAGGACC ATCCGTACAA AACCTGGGCA TATCACGGCA |
| 6001 | GCTACGAAAC CAAACAGACC GGTTCTGCAA GCAGTATGGT TAACGGCGTT GTTCGTCTGC |
| 6061 | TGACCAAACC GTGGGACGTT GTTCCGATGG TTACCCAAAT GGCAATGACC GATACCACCC |
| 6121 | CGTTTGGTCA GCAGCGCGTT TTCAAAGAGA AGGTCGATAC CCGTACCCAA GAACCGAAAG |
| 6181 | AAGGCACCAA GAAGCTGATG AAGATCACCG CTGAGTGGCT GTGGAAAGAA CTGGGCAAGA |
| 6241 | AGAAAACCCC GCGTATGTGT ACCCGCGAAG AATTCACCCG TAAAGTTCGT AGTAACGCTG |
| 6301 | CACTGGGTGC GATTTTCACC GACGAAAACA AGTGGAAGTC TGCACGCGAA GCAGTTGAAG |
| 6361 | ATAGTCGTTT CTGGGAGCTG GTCGACAAAG AACGTAACCT GCATCTGGAA GGTAAGTGCG |
| 6421 | AAACCTGCGT CTACAACATG ATGGGCAAAC GCGAGAAGAA ACTGGGCGAA TTTGGCAAAG |
| 6481 | CGAAAGGCAG TCGCGCTATT TGGTATATGT GGCTGGGCGC ACGTTTTCTG GAATTTGAAG |
| 6541 | CACTGGGCTT CCTGAACGAA GATCACTGGT TTAGCCGCGA AAACAGTCTG TCTGGCGTTG |
| 6601 | AAGGCGAAGG TCTGTATAAA CTGGGCTATA TCCTGCGCGA TGTCAGCAAA AAAGAAGGCG |
| 6661 | GCGCAATGTA TGCAGACGAT ACCGCAGGTT GGGATACCCG TATTACCCTG GAAGACCTGA |
| 6721 | AGAACGAAGA AATGGTCACC AACCACATGG AAGGCGAACA CAAGAAACTG GCGGAAGCGA |
| 6781 | TCTTCAAGCT GACCTACCAG AACAAAGTCG TTCGCGTTCA ACGTCCGACC CCGCGCGGTA |
| 6841 | CCGTTATGGA TATTATTAGC CGTCGCGATC AACGCGGTTC TGGTCAAGTT GGTACCTACG |
| 6901 | GTCTGAACAC CTTCACCAAC ATGGAAGCGC AGCTGATTCG TCAGATGGAA GGCGAAGGCG |
| 6961 | TATTCAAAAG CATCCAGCAT CTGACCGTTA CCGAAGAAAT TGCGGTTCAA AATTGGCTGG |
| 7021 | CACGCGTTCG TCGCGAACGT CTGTCTCGTA TGGCAATTTC TGGCGACGAT TGCGTAGTTA |
| 7081 | AACCGCTGGA TGATCGTTTT GCATCTGCAC TGACCGCTCT GAACGATATG GGCAAAGTCC |
| 7141 | GCAAAGACAT TCAACAGTGG GAACCGAGTC GCGGTTGGAA CGATTGGACC CAAGTTCCGT |
| 7201 | TTTGCAGCCA TCACTTCCAC GAGCTGATCA TGAAAGACGG TCGCGTTCTG GTAGTTCCGT |
| 7261 | GTCGTAATCA AGACGAACTG ATTGGTCGCG CACGTATTTC TCAAGGCGCA GGTTGGTCAC |
| 7321 | TGCGCGAAAC CGCTTGTCTG GGTAAATCTT ACGCACAGAT GTGGAGCCTG ATGTACTTTC |
| 7381 | ATCGTCGCGA TCTGCGTCTG GCAGCAAACG CGATTTGTTC TGCAGTTCCG AGTCATTGGG |
| 7441 | TTCCGACCAG TCGTACCACC TGGAGTATTC ACGCCAAACA CGAGTGGATG ACCACCGAAG |
| 7501 | ATATGCTGAC CGTATGGAAC CGCGTTTGGA TCCAAGAAAA CCCGTGGATG GAAGACAAAA |
| 7561 | CCCCGGTTGA AAGCTGGGAA GAAATCCCGT ATCTGGGTAA ACGCGAAGAT CAGTGGTGCG |
| 7621 | GTAGTCTGAT TGGTCTGACC TCTCGCGCAA CCTGGGCAAA AAACATCCAG ACCGCGATCA |
| 7681 | ACCAGGTCCG TAGCCTGATT GGCAACGAAG AGTATACCGA CTACATGCCG AGCATGAAAC |
| 7741 | GCTTTCGTCG CGAAGAAGAA GAAGCTGGCG TACTGTGGCA TCATCATCAT CATCACTAAT |
| 7801 | GAAAGCTT |

The amino acid sequence of the protein (molecular weight of 104204.4, pl value of 8.75) encoded by this sequence is as shown in SEQ ID NO.4.

2. Construction of DENV-2 NS5 Full-Length Expression Vector

1 μg of the DNA fragment synthesized in step 1 is added to a digestion buffer, digested with Nde l and Hind III endonucleases, 1U each at 16° C. overnight; in another test tube, PET 30a plasmid is digested with Nde I and Hind III. After purifying the digested fragments separately, the two digestion reaction products are subjected to a ligation reaction, i.e., to construct the expression plasmid PET 30a/NS5, which is transformed into E. coli Top 10 competent plasmids for amplification. Sequencing confirms that the genes are inserted correctly and transformed into the expression strain E. coil BL21 (DE3).

3. Expression of DENV NS5 in E. coli

E. coli BL21(DE3)IPET 30a1NS5 colonies are inoculated in a 5 mL LB medium containing kanamycin. After overnight culture, IPTG is added for induction for 4 h; bacteria are collected and ultrasonically lysed; after centrifugation., precipitates of bacterial fragments (treated with Tris base and urea) and the supernatant are separated; after the supernatant passing through a Ni-IDA purification column, the effluent and imidazole eluate are subjected to SDS-PAGE electrophoresis to verify the NSS protein expression and the solubility of the expressed products. The electrophoresis image of FIG I shows that NS5 is mainly expressed in bacterial inclusion bodies.

After confirming the expression of NS5, single colonies of E. coil BL21(DE3)/PET 30a/NS5 are cultured in a 5 mL LB medium. containing kanamycin overnight, and transferred to a 1 L LB medium containing kanamycin (50 μg/mL) the next day; after the bacterial solution is incubated at 37° C. in a shaker to have a turbidity A600>0.6, IPTG is added until the final concentration is 0.5 mM to carry out low temperature induction; after overnight culture at 15° C., bacterial bodies are collected under the conditions that 10,000 g are centrifuged at 4° C. for 15 min, and the precipitates of the bacterial bodies is resuspended in a solution containing 1%

Triton X-100, 1 μg/mL pepstatin A, 1 μg/mL leupeptin and 150 mM NaCl with pH 7.2, and cooled in an ice bath.

4. Purification and Renaturation of DENV NSS Recombinant Protein

The suspension of bacterial bodies is subjected to ultrasonication in an ice bath, and then centrifuged at 12,000 g and 4° C. for 1 h; the precipitates are separated. The precipitates are washed with 50 mM. Tris (pH 8.0) containing 1% Triton X-100, 5 mM. EDTA and 2 mM DTT and 150 InM NaCl solution; after removing the washing solution, the precipitates are dissolved in 20 mM Tris (pH 8.0), 150 mM NaCl, 8 M urea and 20 mM imidazole buffer. After the Ni-IDA agarose purification column is passed through the column as the equilibrium solution, the dissolved protein solution is slowly loaded onto the column, and the non-specific proteins are washed through the column successively with 20 mM, 50 mM, and 100 mM imidazole eluents; the recombinant target protein is eluted using 500 mM imidazole eluent, and the eluent containing the target protein is collected.

The collected purified protein is transferred into a dialysis bag and dialyzed in a buffer containing PBS (pH 7.4), 2 mM EDTA, 4 mM GSH, 0.4 mM GSSG, 0.4 M L-arginine and 2 M urea, and subsequently, further dialyzed in PBS (pH 7.4) containing 10% glycerol for 6 to 8 h. After the renatured target protein solution is sterilized by filtration through a 0.45 μm filter membrane, the concentration is measured, and the renatured target protein solution is cryopreserved at −20° C.

5. Screening of NS5 Protein-Binding Peptides from a Phage Display Peptide Library

The conformational peptide library used for the screening is a random cycloheptapeptide library displayed by M13 phage, purchased from NEWENGLAND BioLabs, USA.

1) The protein solution is diluted with 0.1 M NaHCO₃ to 100 μg/mL. Each time, 0.7 mL of the protein solution is added dropwise to a (φ35 mm polyethylene culture dish, which is gently shaken to soak the dish, which is then coated overnight at 4° C., The coating solution is aspirated and discarded; 2 mL of a blocking solution is added, and left at 4° C. for 2 h; the blocking solution is discarded, and the plate is tap-dried; the plate is washed 6 times with TBST (TBS+0,1%[V/V]Tween−20), and tap-dried each time.

2) Phage (the first round of screening is from a kit containing 10 μL of phage storage solution, about 2×10¹¹ phage particles; thereafter, an amplification and purification solution containing at least 10⁹ phage particles is added each round) is mixed in 0.4 mL of TBST, which is added dropwise to a dish and slowly shaken at room temperature for 50 min; unbound phage is aspirated and discarded, and the plate is tap-dried on a clean paper towel; the dish is washed 10 times with TBST; 0.4 mL of 0.2 mol/L GlycineHCl (pH 2.2, in 1 mg/mL BSA), and shaken slowly for 5 min, then pipetted into a centrifuge tube, neutralized by quickly adding 60 μL of 1 mol/L TrisHCl (pH 9.1) to obtain the eluted phage.

3) The eluted phage is added to 10 mL of the host bacterial Tet-LB culture solution (OD600 to 0.5) and then amplified; after 5 hours of culture, the culture solution is collected to purify the phage, and used for the next round of screening after the titer is measured. 50 blue spots are picked from the plate used to determine the titer after the fifth round of elution and amplified in 1 mL of fresh ER2738 bacterial solution.

6. Determination of the Sequence of Specific Binding Peptides

The preceding amplified 30 phage clones are purified to prepare a single-stranded DNA sequencing template, and sequenced with −96gIII sequencing primers (5′-CCC, TCA, TAG, TCG TAA, CG-3′) to determine the insertion sequence in the PIII protein gene. The measured nucleotide fragments of the 30 clones are translated into amino acid sequences, and it has been found that the amino acid sequences of the phage display peptides eluted in the last round have similarities, wherein the amino acid sequences of 15 clones are completely identical, and all are KHGHHRH, i.e., Lys-His-Gly-His-His-Arg-His.

Detai Bio-Tech (Nanjing) Co., Ltd. was commissioned to synthesize the cyclized peptide of this sequence, which is formulated into a 1 g/L mother liquor, and added to the newly infected dengue virus-infected cells according to the concentration gradient. The cell changes are observed day by day, and the synthesized peptide is found to have a significant protective effect against viral infection, and the protective effect is of a dose-effect relationship. The beneficial effects of the disclosure are as follows:

The disclosure uses genetic engineering and phage display peptide library technology to obtain the full-length protein of dengue virus NS5, and accordingly, selects an oligopeptide that can inhibit the replication of dengue virus for all types of dengue virus, which provides a new way to treat dengue viruses and effectively avoids the problem of being only effective against one type of virus, but easy to produce or anravate the infection of heterotypic viruses.

### BRIEF DESCRIPTION OF THE DRAWINGS

FIG. 1 is an SDS-PAGE electrophoresis image of the full-length expression of the dengue virus NS5 protein according to the disclosure; the SDS-PAGE electrophoresis image shows that NS5 is mainly expressed in bacterial inclusion bodies, wherein M: protein molecular weight standard; 1: precipitate after bacterial lysis and centrifugation; 2: supernatant after bacterial lysis and centrifugation; 3: effluent after passing the supernatant through a Ni-IDA purification column; 4: 50 inM imidazole eluent after the supernatant is passed through the purification column; 5-6: 100 mM imidazole eluent after the supernatant is passed through the purification column; 7: 500 mM imidazole eluent after the supernatant is passed through the purification column (4-7 are respectively 50 mM, 100 mM, 100 mM, and 500 mM imidazole eluents after the supernatant is passed through the Ni-IDA purification column).

FIG. 2 shows the Western blotting identification of the recombinant expression product of the dengue virus NSS protein (104 kDa); wherein M: protein molecular weight standard; 1: supernatant of whole strain lysate of expression. strain; 2: precipitate of whole strain lysate of expression strain; 3: inclusion body solution.

FIG. 3 shows the protective effect of the synthesized cyclic peptide in the example on virus-infected cells; wherein I: cells without addition of the synthesized cyclic peptide; 2: cells with addition of a low concentration of the synthesized peptide; 3: infected cells with addition of a high concentration of the synthesized peptide; 4: non-infected control cells.

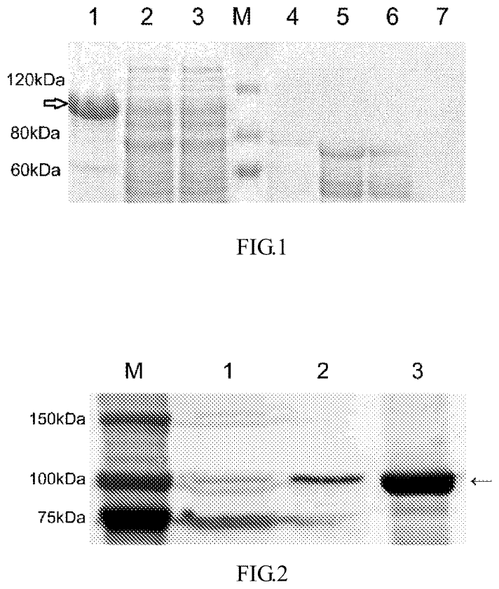

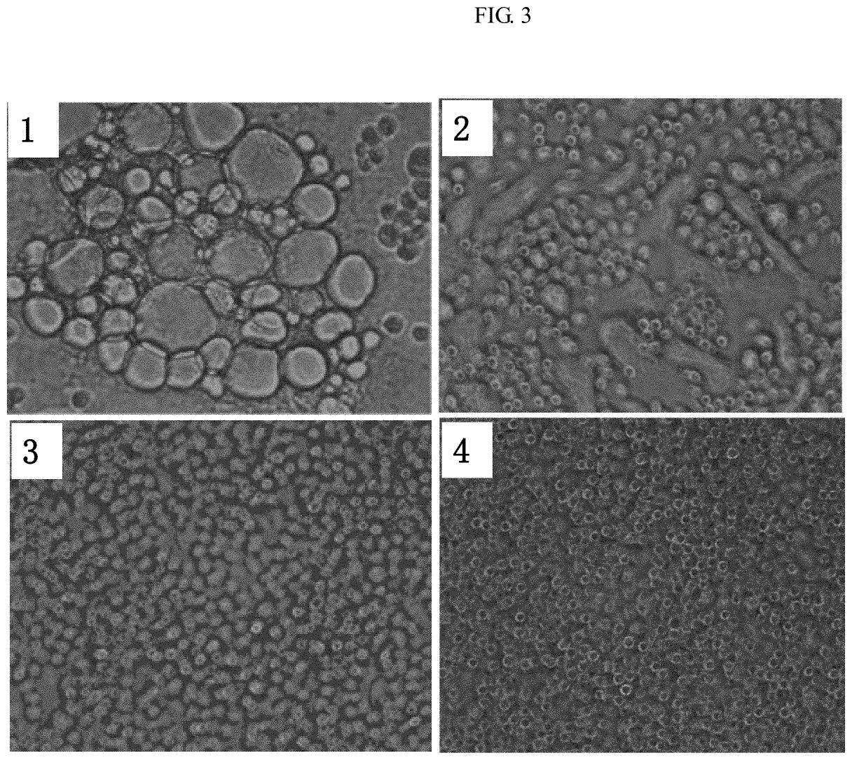

### DESCRIPTION OF THE EMBODIMENTS

This disclosure is further explained below with reference to the example. It should be understood that the following example is for illustrative purposes only and is not intended to limit the scope of the disclosure. Those skilled in the art can make various modifications and substitutions to the disclosure without departing from the principle and spirit of the disclosure.

Unless otherwise specified, the experimental methods used in the following example are all conventional methods.

The materials, reagents and the like used in the following example all can be obtained from commercial sources, unless otherwise specified.

### EXAMPLE 1

This example is used to illustrate the preparation and frictional study of the synthesized peptide of the disclosure.

1. The oligopeptide can be synthesized by constructing an expression vector through gene recombination. The codon sequence is AAG AAT ACT CTT CAT ACG TTT or AAG CAT GGT CAT CAT CGT CAT. These are also nucleotide sequences obtained by sequencing during the phage peptide library screening. Alternatively, a nonapeptide with the sequence CKHGHHRHC is synthesized by chemical methods, and the cysteines at both ends are used to cyclize the oligopeptide.

2. Dry powder of the synthesized peptide is diluted to a concentration of 1 with a DMEM cell culture medium. The cryopreserved virus is expanded and cultured, and a 500-fold TCID 50/mL of dengue virus suspension is prepared.

3. C6/36 cells are cultured on a microplate at 28° C., and the original culture solution on the microplate is aspirated off when the cell density reaches about 70% 100 μL of DMEM cell growth and maintenance solution is added to each well in the first row of the culture plate, which are non-infected and pepetide-free control cells; 25 μL of peptide solution is added to each well in. the second row, and 50 82 L of peptide solution is added to each well in the third row, which are non-infected control cells with the addition of pepetides; 25 μL of virus solution is added to each well in the fourth to eighth rows, and the cell culture plate is placed in an incubator for 1 h to allow the virus to adsorb cells. The fourth row is free of peptide solution, and each of the fifth to eighth rows is loaded with peptide solution by 5 μL/well, 10 μL/well, 20 μL/well and 40 μL/well respectively; the pores with a total liquid amount of less than 100 μl in the culture system is supplemented to 100 μL, and the culture plate is cultured in a carbon dioxide incubator.

4. Cell lesions are observed for 4 days, and the number of cell wells and the degree of lesions of cytopathic cells in each row are recorded. The degree of cell lesion is divided into: 0, no cell lesion; I, 0 to 25% of the cells have lesions; II, 25 to 50% of the cells have lesions; III, 50 to 75% of the cells have lesions; IV, 75 to 100 % of the cells have lesions.

The results show that the cells in the 36 culture wells in the to 1^{st} to 3^{rd} rows grow well; the cells in the 12 wells of the 4^{th} row without the addition of the synthesized peptide all develop lesions, wherein 9 wells have a lesion degree of IV and 3 wells have a lesion degree of III; cells in 12 wells in the 5^{th} row with the addition of 5 μL/well of peptide solution have lesions, and the degrees of which are II to III; 9 wells of cells in the 6^{th} row with the addition of 10 μL/well of peptide solution have lesions, and the degrees of which are I to II; 6 wells of cells in the 7^{th} row with the addition of 20 μL/well of peptide solution have lesions, and the degrees of which are I to II; only 3 wells of cells in the 8^{th} row with the addition of 40 μL/well of peptide solution have lesions, and the degree of which is I; continued culture reveals the return to normality.

The above cell experiments show that the synthesized peptide of the disclosure has no adverse effect on cell growth at high concentrations; the synthesized peptide has a significant protective effect on cells against virus infection, and the protective effect exhibits a dose-effect relationship.

It should be understood that the technical solution obtained after proportionally increasing or reducing the amount of the reagents or raw materials used in the above example is substantially the same as that of the above example.

Although this disclosure has been described in detail with the general descriptions and specific embodiments, it is obvious to those skilled in the art that modifications or improvements can be made to the present invention on the basis of the this disclosure. Therefore, these modifications or improvements made without departing from the spirit of this disclosure belong to the scope of protection of this disclosure.

### INDUSTRIAL APPLICABILITY

The disclosure provides an oligopeptide having the function of inhibiting dengue virus replication and the application thereof. The amino acid sequence of the oligopeptide provided by the disclosure is KHGHHRH, i.e., Lys-His-Gly-His-His-Arg-His. The oligopeptide has a high specific affinity for NS5, and has a highly effective inhibitory effect on dengue virus replication. The antiviral effect thereof is not limited to DENV 2, and it also has a significant inhibitory effect on the replication of type I, type 3, and type 4 dengue viruses. One cysteine is added to each end of the oligopeptide sequence, and the oligopeptide can be cyclized by the cysteines at both. ends to form a cyclic peptide. The obtained cyclic peptide has enhanced effect of inhibiting dengue virus replication, and can be used for specific treatment of dengue virus infection, and has good economic value and application prospect.

What is claimed is:

1. An oligopeptide having the function of inhibiting dengue virus replication, characterized in that the amino acid sequence of the oligopeptide is KHGHHRH.

2. Use of the oligopeptide according to claim 1 in the manufacture of a medicament for treating dengue virus infection.

3. Use of the oligopeptide according to claim 1 in the manufacture of a medicament for inhibiting dengue virus replication.

4. A pharmaceutical composition, characterized in comprising the oligopeptide according to claim 1.

5. The pharmaceutical composition according to claim 4, characterized in that the oligopeptide may be cychzed to form a cyclic peptide.

6. A oligopeptide having the function of inhibiting dengue virus replication, characterized in that the oligopeptide is a tripeptide, tetrapeptide, pentapeptide or hexapeptide fragment in the oligopeptide according to claim 1.

7. Use of the oligopeptide according to claim 6 in the manufacture of a medicament for treating dengue virus infection.

8. Use of the oligopeptide according to claim 6 in the manufacture of a medicament for inhibiting dengue virus replication.

9. A pharmaceutical composition, characterized in comprising the oligopeptide according to claim 6.
